(19) United States
(12) Patent Application Publication
(10) Pub. No.: US 2005/0192650 A1
(43) Pub. Date: Sep. 1, 2005

(54) Hand-held device for ameliorating skin imperfections using LED light emissions

(76) Inventor: Gloria Martel, Los Angeles, CA (US)

Correspondence Address: MCANDREWS HELD & MALLOY, LTD, 500 WEST MADISON STREET, SUITE 3400, CHICAGO, IL, 60661, US

(21) Appl. No.: 10/929,953

(22) Filed: Aug. 30, 2004

Related U.S. Application Data
(60) Provisional application No. 60/547,888, filed on Feb. 26, 2004.

(51) Int. Cl. A61N 5/06

(52) U.S. Cl. 607/88

(57) ABSTRACT

A hand-held device for ameliorating skin imperfections includes a generally longitudinally extending housing, a lens mounted at one end of the housing, the lens having a plurality of openings formed therein, and a plurality of light-emitting diodes disposed in the interior of the housing. The light-emitting diodes are arranged such that light emissions from the diodes are transmitted substantially unimpeded from the interior of the housing. Directing the light emissions upon an area of skin ameliorates imperfections present in the skin area.

### CROSS-REFERENCE TO RELATED APPLICATION(S)

This application relates to and claims priority benefits from U.S. Provisional Patent Application Ser. No. 60/547,888 filed Feb. 26, 2004, entitled “Skin Lift Light”. The '888 application is hereby incorporated by reference herein in its entirety.

### FIELD OF THE INVENTION

The present invention relates to a hand-held device for improving the condition of skin. In particular, the present invention relates to a hand-held device that directs light emissions from light-emitting diodes (LED) onto the skin to improve the user's skin complexion and ameliorate wrinkles, acne and other skin surface imperfections.

### BACKGROUND OF THE INVENTION

The application of non-invasive and non-thermal light to the outer layer of the skin has been shown to provide significant benefits in the area of cosmetic skin treatment and rejuvenation. Effective therapeutic techniques for the skin can increase and stimulate blood circulation in the area of the skin being treated.

Light-emitting diode (LED) light therapy devices can improve and stimulate blood circulation in the skin via the absorption of light by the skin and underlying tissues, thereby stimulating cell metabolic activity, promoting muscle relaxation, and accelerating tissue repair mechanisms. The result of directing LED light emissions onto skin is the diminution of toxins and metabolic by-products from ailing skin tissue, while at the same time promoting the flow of nutrients to the affected tissue through increased blood flow.

Persons seeking to improve their skin complexion using light therapy have generally done so in specialized physicians offices. Such specialized treatments involve considerable expense and time commitments. More and more people wish to conveniently and economically acquire a youthful skin appearance, and are seeking techniques that smooth wrinkles, reduce or eliminate acne, and rejuvenate the skin, all in the privacy and convenience of the home and at low cost.

LED therapeutic techniques involving emission of light at a specific wavelength have been shown effective in reducing or eliminating acne. In the case of acne, light absorption by the affected skin tissue cells induces changes in cell membrane permeability, which in turn promotes the influx of protons and the dissipation of pH gradients across cell membranes. The combination of these factors results in the restoration of a more favorable homeostatic state to the philosebaceous tissue involved, as well as the creation of a microenvironment that discourages the formation of papules and pustules.

Professor Mester of Budapest University has carried out experiments with animal and human tissue cells to investigate the effect of light emission on the cell. According to Professor Mester's experiments, it is the monochromatic property of light, and not its coherence, that influences cellular DNA to more effectively utilize lipoprotein in light-treated areas. The result of monochromatic light treatment is improved performance of the cells' metabolic processes, and in the case of skin tissue results in increased production of collagen and elastin. Professor Mester's experiments have demonstrated that monochromatic light, although non-coherent, has the ability to smooth wrinkles and reduce acne without significant damage to the epidermis.

Niels Ryberg Finsen, M.D., a 1903 Nobel laureate in medicine, was recognized for his contribution to the treatment of diseases, especially lupus vulgaris, with concentrated light radiation. Dr. Finsen has been credited with discovering the curative power of different colors of light, which led to his founding the medical science of phototherapy. Dr. Finsen also devised a treatment for smallpox using red light (1893) and a light-based treatment for lupus (1895).

Harry T. Whelan, M.D., of the Medical College of Wisconsin, has published results of studies carried out in connection with NASA's light-emitting diode medical program. Those and other medical research studies support the regenerative effects of monochromatic light on ameliorating skin imperfections.

### SUMMARY OF THE INVENTION

A hand-held device for ameliorating skin imperfections comprises:
(a) a generally longitudinally extending housing;
(b) a lens mounted at one end of the housing, the lens having a plurality of openings formed therein;
(c) a plurality of light-emitting diodes disposed in the interior of the housing, the light-emitting diodes arranged such that light emissions from the diodes are transmitted substantially unimpeded from the interior of the housing.
   In operation, directing the light emissions upon an area of skin ameliorates imperfections present in the skin area.

In a preferred hand-held light-emitting device, each of the diodes emits light has a wavelength between about 525 nm and about 690 nm. The plurality of light-emitting diodes preferably comprises five light-emitting diodes.

The device preferably further comprising a three-position switch. In a first switch position, all of the light-emitting diodes are actuated. In a second switch position, a portion of the light-emitting diodes is actuated. In a third switch position the light-emitting diodes are deactuated.

In a preferred embodiment, the plurality of light-emitting diodes comprises five light-emitting diodes, all five of the light-emitting diodes are actuated in the first switch position, and two of the light-emitting diodes are actuated in the second switch position.

A method of ameliorating skin imperfections employs a hand-held device having a generally longitudinally extending housing. A lens is mounted at one end of the housing. The lens has a plurality of openings formed therein. The housing has a plurality of light-emitting diodes disposed in the interior of the housing. The light-emitting diodes are arranged such that light emissions from the diodes are transmitted substantially unimpeded from the interior of the housing. The method comprises directing the light emissions upon an area of skin such that imperfections present in the skin area are ameliorated.

### BRIEF DESCRIPTION OF THE DRAWINGS

FIG. 1 is a top view, partially in section, of the present hand-held device for improving the condition of skin using LED light emissions.

FIG. 2 is a bottom view of the hand-held LED light-emitting device shown in FIG. 1.

FIG. 3 is a side view, partially in section, of the hand-held LED light-emitting device shown in FIG. 1.

FIG. 4 is a front elevation view of the lens portion of the hand-held LED light-emitting device shown in FIG. 1.

FIG. 5 is a front perspective view of the lens portion of the hand-held LED light-emitting device shown in FIG. 1.

FIG. 6 is a top sectional view of the lens portion of the hand-held LED light-emitting device, taken in the direction of arrows A-A in FIG. 4.

FIG. 7 is a side sectional view of the lens portion of the hand-held LED light-emitting device, taken in the direction of arrows B-B in FIG. 4.

FIG. 8 is a circuit diagram of the electronic components incorporated in the present hand-held device for improving the condition of skin using LED light emissions.

### DETAILED DESCRIPTION OF PREFERRED EMBODIMENT(S)

Turning to FIGS. 1-3, a hand-held LED light-emitting device 10 for improving the condition of skin includes a housing 12, preferably formed of moldable plastic material, and a lens 14, preferably formed of moldable transparent plastic material. A plurality of light-emitting diodes, one of which is designated in FIG. 1 as LED 16, is arranged adjacent the interior facing surface of lens 14. The illustrated embodiment contains five LEDs.

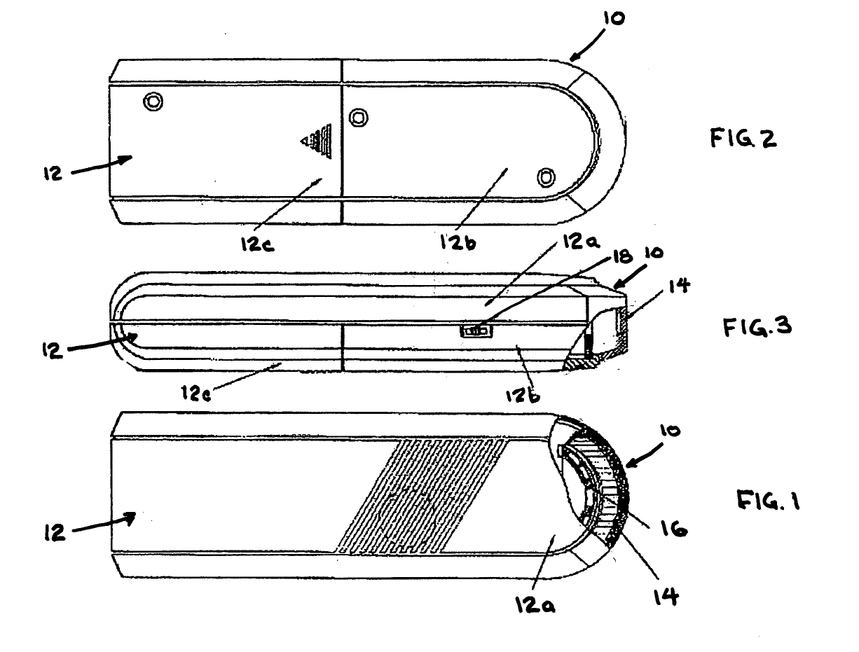

As further shown in FIGS. 1 and 2, a side-mounted three-position switch 18, in a first position (rightmost position in FIG. 2), actuates all five of the LEDs; in a second position (leftmost position in FIG. 2), actuates only two of the LEDs; and in a third position (middle position in FIG. 2) turns off all of the LEDs.

As shown in FIGS. 2 and 3, housing 12 has three parts: a fixed top portion 12a, a fixed bottom portion 12b and a slidable bottom portion 12c. Portion 12c is slidable to provide access to the battery (a 9-volt battery in the illustrated embodiment) mounted in the interior of device 10.

In the illustrated embodiment, the LEDs emit light having wavelengths between about 525 nm and about 690 nm.

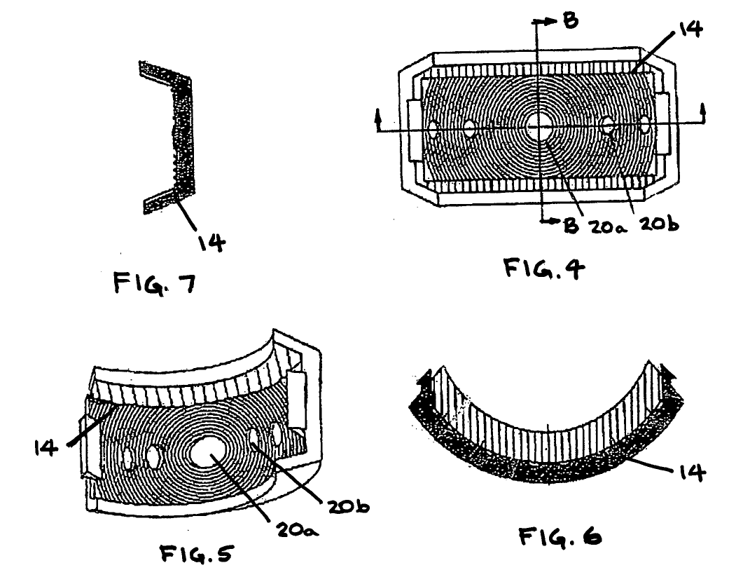

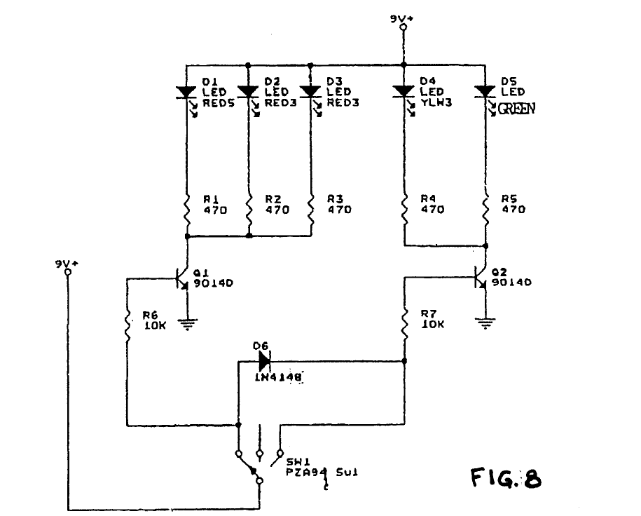

FIGS. 4-7 illustrate the conformation of lens 14, and in particular the arrangement of the various facets of lens 14, which are depicted in FIGS. 4-7 using hatched and solid shading. As shown in FIGS. 4 and 5, lens 14 has a plurality of substantially transparent windows 20, two of which are shown in FIGS. 4 and 5 as windows 20a and 20b, formed therein to permit light emissions from the LEDs to be transmitted substantially unimpeded from the interior of device 10.

In FIG. 8, the illustrated circuit diagram depicts a preferred configuration of the electronic components of device 10.

The following is a description of a preferred technique for using the present hand-held LED light-emitting device:
(a) Wash face and skin to be treated to remove oils and topical substances such as makeup.
(b) Starting at the back of the neck, just below the hairline; hold the light apparatus just above or against the skin for a length of time needed to ameliorate the skin condition involved.
(c) Slowly move the apparatus down the neck to the shoulder, around to the front of the neck, and then up and over the face.

In the case of loose skin, the LED light emissions should be directed on the affected skin area for a longer period of time to more aggressively ameliorate skin conditions such as sagging jaw lines, crow's feet, deep smile lines, and the like. In the case of acne, LED light emissions should be directed on the affected skin area over multiple treatments per day of several minutes each until the condition remediates.

While particular elements, embodiments and applications of the present invention have been shown and described, it will be understood, of course, that the invention is not limited thereto since modifications may be made by those skilled in the art without departing from the scope of the present disclosure, particularly in light of the foregoing teachings.

1. A hand-held device for ameliorating skin imperfections comprising:
(a) a generally longitudinally extending housing;
(b) a lens mounted at one end of said housing, said lens having a plurality of openings formed therein;
(c) a plurality of light-emitting diodes disposed in the interior of said housing, said light-emitting diodes arranged such that light emissions from said diodes are transmitted substantially unimpeded from the interior of said housing;
whereby directing said light emissions upon an area of skin ameliorates imperfections present in said skin area.

2. The device of claim 1 wherein each of said diodes emits light having a wavelength between about 525 nm and about 690 nm.

3. The device of claim 1 wherein said plurality of light-emitting diodes comprises five light-emitting diodes.

4. The device of claim 1 further comprising a three-position switch, a first of said positions actuating all of said light-emitting diodes, a second of said positions actuating a portion of said light-emitting diodes, and a third of said positions deactuating said light-emitting diodes.

5. The device of claim 4 wherein said plurality of light-emitting diodes comprises five light-emitting diodes, all five of said light-emitting diodes are actuated in said first switch position, and two of said light-emitting diodes are actuated in said second switch position.

6. A method of ameliorating skin imperfections using a hand-held device having a generally longitudinally extending housing having a lens mounted at one end thereof, said lens having a plurality of openings formed therein, said housing having a plurality of light-emitting diodes disposed in the interior thereof; said light-emitting diodes arranged such that light emissions from said diodes is transmitted substantially unimpeded from the interior of said housing, the method comprising:
directing said light emissions upon an area of skin, whereby imperfections present in said skin area are ameliorated.
